(19) United States
(12) Patent Application Publication
(10) Pub. No.: US 2011/0112044 A1
(43) Pub. Date: May 12, 2011

(54) NOVEL USES OF D-MANNOPYRANOSE DERIVATIVES

(75) Inventors: Jean-Louis Monteron, Lauret (FR); Veronique Montero, Lauret (FR); Jean-Pierre Moles, Cournonsec (FR); Pascal De Santa Barbara, Fabregues (FR); Caroline Clavel, Assas (FR); Bernard Jover, Montpellier (FR)

(73) Assignee: CENTRE NATIONAL DE LA RECHERCHE SCIENTIFIQUE, Paris (FR)

(21) Appl. No.: 12/991,631

(22) PCT Filed: May 5, 2009
(86) PCT No.: PCT/FR2009/000525

§ 371 Date: Jan. 26, 2011

(30) Foreign Application Priority Data
May 7, 2008 (FR) 08 02537

(51) Int. Cl. A61K 31/7028; A61K 31/7034; A61K 31/7042; A61P 19/02

(52) U.S. Cl. 514/27; 514/25

(57) ABSTRACT

The invention relates to the use of mannose-6-phosphate (M6P) and of certain derivatives thereof for controlling angiogenesis and ligament regeneration and/or cartilage reconstruction. The MP6 and certain derivatives thereof can particularly be used for preparing a pharmaceutical composition used for ligament regeneration and/or cartilage reconstruction.

The present invention relates to the use of mannose-6-phosphate (M6P) and of certain derivatives thereof for controlling angiogenesis and ligament regeneration and/or cartilage reconstruction. M6P and certain derivatives thereof may especially be used for the preparation of a pharmaceutical composition intended for ligament regeneration and/or cartilage reconstruction.

Many pathologies have been described as having a component or stage linked to the phenomenon of angiogenesis. Mention may be made, inter alia, of numerous cancers, diabetes-related retinopathies, atherosclerosis, arthrosis, rheumatoid arthritis, psoriasis and inflammatory pathologies or pathologies associated with delayed wound healing.

Angiogenesis is a mechanism of neovascularization stemming from a preexisting capillary network. The budding of small vessels, the capillaries, from preexisting vessels, arises in the best case during development of the embryo and implantation of the placenta, when it is the case of healing a wound, or of overcoming the obstruction of a vessel; but also, in the worst case, in cancers (growth of tumors and development of metastases), rheumatoid arthritis, certain ophthalmological diseases such as diabetic retinopathy or age-related macular degeneration, etc. For all these processes, the general scheme remains the same. Activation of the endothelial cells leads to degradation of the basal membrane and of the surrounding extracellular matrix. The directed migration is followed by a proliferative phase. The cells then differentiate into a structure of capillary type to form a vascular network necessary for the growth of the tissues. In recent years, it has become clear that angiogenesis is not controlled by a single factor, but by a balance of inducers and inhibitors produced by normal or tumoral cells. Among these factors, polypeptides such as fibroblast growth factor-2 (FGF-2) and vascular endothelial growth factor (VEGF) have appeared as being key regulators of angiogenesis.

Many molecules have been studied for their inhibitory or activating effect on angiogenesis.

As regards angiogenesis inhibition, a recent conceptual revolution in cancer treatment consists in targeting the vascular network that irrigates a tumor. It is now well established that the development of intratumoral or peritumoral vascularization is a key event both for the growth of a tumor and for metastatic dissemination via the blood system. In December 2005, the English scientific review Nature, which devoted its issue to angiogenesis, counted more than 300 inhibitors, including 80 undergoing clinical trials. However, the first medicaments tested—angiostatin, endostatin, interferons, metalloprotease matrix inhibitors etc.—were disappointing. Among more recent molecules, mention may be made of bevacizumab. When injected into a patient, it neutralizes a type of VEGF circulating in the capillaries or diffused in the tumor, VEGF-A. Its first indication was in 2004 for metastatic colorectal cancer, in combination with chemotherapy. It is now in the course of clinical trials for combating metastatic kidney cancer, lung cancer and breast cancer. However, it is observed that it increases the risk of hypertension and hemorrhaging. Mention may also be made of sunitinib and sorafenib, which have the advantage of allowing formulation in the form of oral tablets and which lead to encouraging therapeutic results. They also have the drawback of giving rise to a few side effects, such as hypertension, fatigue or skin problems.

Thus, the Inventors have discovered that mannose-6-phosphate and certain selected derivatives thereof as will be described hereinbelow (compounds of formula (I)) have angiogenesis-inhibiting activity, and allow ligament regeneration and/or cartilage reconstruction.

One subject of the present invention is thus the use, as an active principle, of at least one compound of formula (I) below:

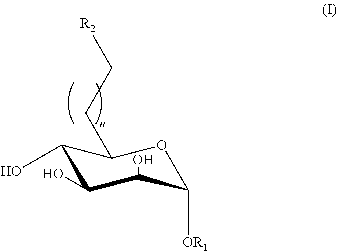

in which:
R₁ represents a linear or branched C₁-C₄ alkyl radical; an alkyl radical comprising one or more functional groups chosen from hydroxyl, amine, thiol, carboxyl, azide and nitrile groups; a saturated or unsaturated C₃-C₆ hydrocarbon-based ring; a saturated or unsaturated C₃-C₆ hydrocarbon-based ring comprising one or more functional groups chosen from hydroxyl, amine, C₁-C₄ alkyl, thiol, carboxyl, azide and nitrile groups; a saturated or unsaturated heterocycle comprising at least one heteroatom chosen from oxygen, nitrogen and sulfur atoms;
n is an integer equal to 0 or 1,
R₂ is chosen from the following groups (G₁) to (G₄):

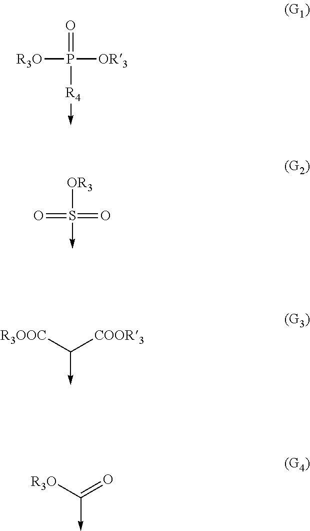

in which:
R₃ and R′₃, which may be identical or different, represent a hydrogen or sodium atom;
R₄ represents an oxygen or sulfur atom, and
the arrow represents the point of attachment of the group to the carbon atom bearing R₂,

for the preparation of a pharmaceutical composition for ligament regeneration and/or cartilage reconstruction.

According to the invention, among the C₁-C₄ alkyl radicals mentioned for R₁, the methyl radical is particularly preferred.

Among the functionalized alkyl radicals cited for R₁, mention may be made in particular of C₁-C₄ mono- and dihydroxyalkyl, C₁-C₄ mono- and diaminoalkyl, C₁-C₄ mono- and and dithioalkyl and C₁-C₄ mono- and dicarboxyalkyl radicals.

Among the hydrocarbon-based rings mentioned for R₁, mention may be made in particular of cyclopropane, cyclobutane, cyclopentane, cyclohexane, phenyl and benzyl rings.

Among the heterocycles mentioned for R₁, mention may be made in particular of oxadiazole, triazole, oxazole, isoxazole, imidazole, thiadiazole, pyrrole, tetrazole, furan, thiophene, pyrazole, pyrazoline, pyrazolidine, triazole, isothiazole, pyridine, pyrimidine, piperidine, pyran, pyrazine and pyridazine rings. In the compounds of formula (I) above, when n=0, R₂ is preferably chosen from the groups G₃ and G₄ and when n=1, R₂ is preferably chosen from the groups G₁ and G₂.

According to one preferred embodiment of the invention, the compounds of formula (I) are chosen from those in which R₂ represents a group G₁ or G₃ as defined above in which R₃ and R′₃ are identical and represent a sodium atom and from those in which R₂ represents a group G₂ or G₄ as defined above in which R₃ represents a sodium atom.

Among the compounds of formula (I) above, mention may be made in particular of:
methyl D-mannopyranoside 6-phosphate, also known under the trivial name mannose-6-phosphate (M6P) in the literature;
methyl (disodium) D-mannopyranoside 6-phosphate;
methyl 6,7-dideoxy-7-sodiumsulfonato-D-manno-heptopyranoside;
(methyl 6,7-dideoxy-D-mannoheptopyranoside)-uronic acid; and
methyl 6-deoxy-6-malonate-D-mannopyranoside.

Among these compounds, methyl D-mannopyranoside 6-phosphate (M6P), methyl (disodium) D-mannopyranoside 6-phosphate and methyl 6,7-dideoxy-7-sodiumsulfonato-D-mannoheptopyranoside are particularly preferred.

Mannose-6-phosphate and some of the compounds of formula (I) listed above are known per se and have already been proposed in the pharmaceutical field, especially for improving skin wound healing while at the same time reducing the formation of unsightly scars (Clavel, C. et al., Il Farmaco, 2005, 60, 721-725). However, they have never yet been used therein and no activity of these compounds on ligament regeneration and/or cartilage reconstruction has yet been described.

As has been seen previously, the compounds of formula (I) in accordance with the present invention have inhibitory activity on angiogenesis and activity on ligament regeneration and/or cartilage reconstruction. They may consequently be used for the preparation of a pharmaceutical composition for ligament regeneration and/or cartilage reconstruction. Specifically, during ligament regeneration or cartilage reconstruction, implants formed from biocompatible polymers containing ad hoc cells are generally used. In this case, it is desirable to prevent vascularization of the implant so as to maintain an acellular material. Thus, for this application, the pharmaceutical composition is preferably in the form of a polymeric biomaterial containing at least one compound of formula (I).

The pharmaceutical composition of the invention as defined above comprises, in addition to the compound of formula (I), at least one pharmaceutically acceptable excipient.

A person skilled in the art will select one or more pharmaceutically acceptable excipients as a function of the route of administration of the pharmaceutical composition. Needless to say, a person skilled in the art will take care at the time to ensure that the excipient(s) used are compatible with the intrinsic properties associated with the composition in accordance with the present invention.

In addition, the form of the medicament or of the pharmaceutical composition (for example a solution, a suspension, an emulsion, tablets, gel capsules, suppositories, a polymeric biomaterial, etc.) will depend on the chosen route of administration.

Thus, for the purposes of the present invention, the medicament or pharmaceutical composition may be administered via any appropriate route, for example orally, locally, systemically, intravenously, intramuscularly or mucosally, or alternatively using a patch or a polymeric biomaterial.

As nonlimiting examples of excipients that are suitable for oral administration, mention may especially be made of talc, lactose, starch and derivatives thereof, cellulose and derivatives thereof, polyethylene glycols, acrylic acid polymers, gelatin, magnesium stearate, animal, plant or synthetic fats, paraffin derivatives, glycols, stabilizers, preserving agents, antioxidants, wetting agents, anticaking agents, dispersants, emulsifiers, flavor enhancers, penetrants, solubilizers, etc.

The techniques for formulating and administering medicaments and pharmaceutical compositions are well known in the art under consideration herein, and a person skilled in the art may especially refer to the latest edition of Remington's Pharmaceutical Sciences.

The compounds of formula (I) may be readily prepared, from a D-mannopyranoside of formula (II) defined below, by nucleophilic displacement of the corresponding cyclic sulfate precursor of formula (IV), by analogy with the method described, for example, by Van der Klein P. A. M. et al., Carbohydr. Res., 1992, 224, 193-200 followed by deprotection of the hydroxyl radicals borne by the saccharide unit, according to reaction scheme A below:

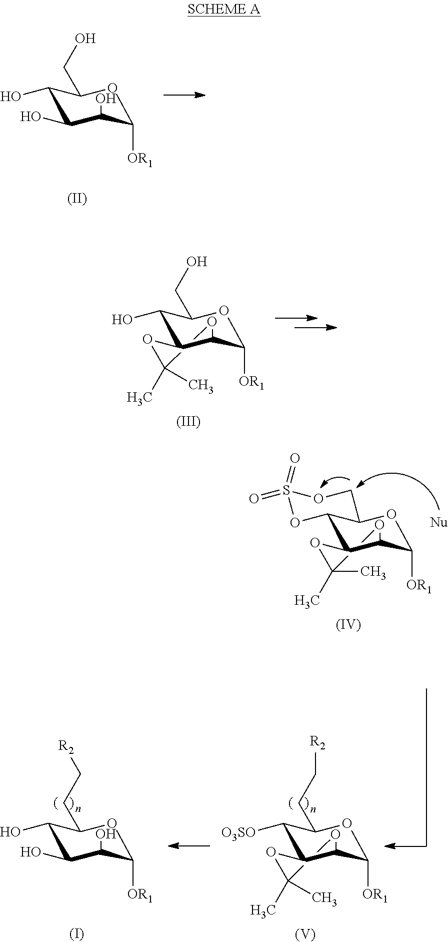

in which R₁, R₂ and n have the same meaning as indicated above for the compounds of formula (I) and Nu represents a nucleophilic group corresponding to the group R₂ that it is desired to introduce.

This method corresponds to an adaptation of the method described in the article by Khanjin N. A. et al., Tetrahedr. Lett., 2002, 43, 4017-4020.

The cyclic sulfate of formula (IV) prepared according to this process may be stored for several months at room temperature in the form of a white powder, without observing any decomposition. The pure intermediates of the monosulfate salts may be readily separated from the unreacted nucleophilic groups and from the other impurities by partition between water and a solvent such as dichloromethane before the deprotection step. The simultaneous and quantitative cleavage of the cyclic monosulfate and isopropylidene groups of the compounds of formula (V) may be performed on an ion-exchange resin such as an Amberlyst-15 (H+) resin, which allows deprotection of the cyclic monosulfate group in 10 to 30 minutes and that of the isopropylidene group in 3 to 5 hours at room temperature in a methanol/tetrahydrofuran mixture. All the compounds of formula (I) prepared according to this process may be obtained in a yield of between 60 and 95%.

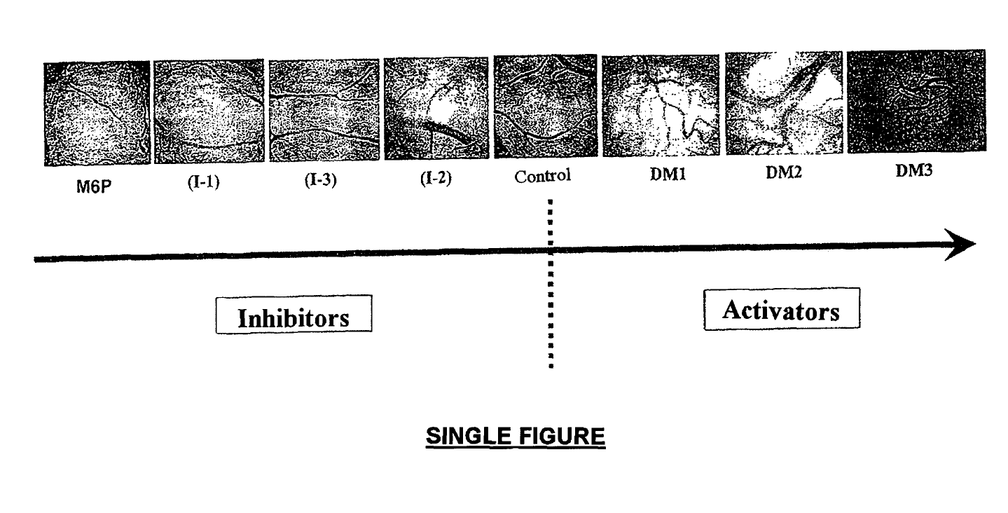

Besides the preceding provisions, the invention also comprises other provisions that will emerge from the description that follows, which refers to examples of preparation of the compounds of formula (I) in accordance with the invention, and also to an example of demonstration of the angiogenesis-inhibiting activity of the compounds of formula (I) relative to other D-mannopyranose derivatives not corresponding to formula (I) and thus not forming part of the invention, and also to the attached FIG. 1 which shows photos of the vascularization of chick embryos after culturing in the presence of 6 mg/ml of different compounds of formula (I) in accordance with the invention compared with three D-mannopyranoside derivatives (DM) having pro-angiogenic activity and thus not forming part of the invention (DM1: methyl 7-amino-6,7-dideoxy-α-D-mannopyranoside; DM2: methyl 6-azido-6-deoxy-α-D-mannopyranoside and DM3: methyl 7-disodiumphosphonato-6,7-dideoxy-α-D-mannoheptopyranoside).

It should be understood, however, that these examples are given purely for the purpose of illustrating the invention, of which they do not in any way constitute any limitation.

### EXAMPLE 1

### Preparation of Methyl 6,7-Dideoxy-7-Sodiumsulfonato-α-d-mannoheptopyranoside (compound of Formula I-1)

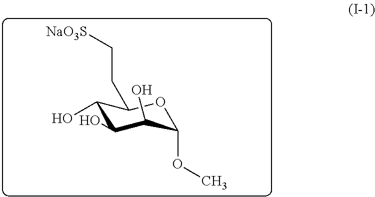

1) First Step: Preparation of Methyl 2,3-O-isopropylidene-4,6-O-(cyclic sulfate)-α-D-mannopyranoside (Compound 3)

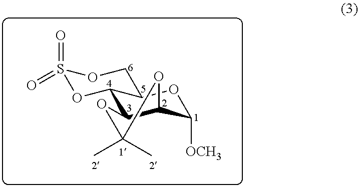

Compound (3) was obtained in two substeps, without intermediate purification, via the corresponding sulfite (2).

### 1-a) Preparation of the Corresponding Sulfite (2)

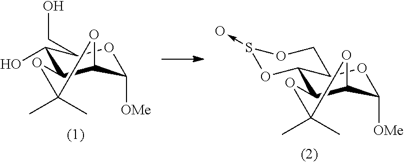

3.79 g (16.18 mmol; 1 eq.) of methyl 2,3-O-isopropylidene-α-D-mannopyranoside (1) and 6.75 mL (48.54 mmol; 3 eq.) of triethylamine were dissolved in 75 mL of dichloromethane (CH₂Cl₂). The mixture was cooled to 0° C. and 1.3 mL (17.80 mmol; 1.1 eq.) of thionyl chloride (SOCl₂) were added slowly. The white precipitate of triethylammonium chloride formed instantaneously, and the reaction mixture gradually turned yellow, then brown, over 5 to 10 minutes. Thin-layer chromatography (TLC) was then performed using as mobile phase a mixture of petroleum ether (PE) and ethyl acetate (EtOAc) (8/2 v/v). The results of this TLC then indicated that no more starting material remained (Rf=0) and that the desired sulfite had been obtained in the form of two diastereoisomers (Rf=0.45 and 0.60). The reaction mixture was then filtered and the organic phase was washed with distilled water, 1N hydrochloric acid (HCl) solution, and again with distilled water. It was dried over sodium sulfate (Na₂SO₄), filtered and concentrated to give a slightly brown solid, which was reused in reaction immediately.

### 1-b) Oxidation of the Sulfite (2) to the Sulfate (3)

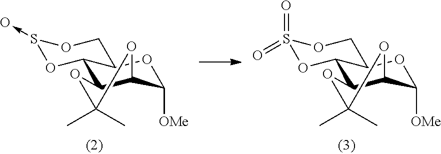

The crude sulfite (2) obtained above in substep 1-a) (16.18 mmol; 1 eq. theoretically) was dissolved in 60 mL of a solution composed of a mixture of CH₂Cl₂ and acetonitrile (CH₃CN) (1/1 v/v), followed by successive addition of 3.8 g (17.80 mmol; 1.1 eq.) of sodium metaperiodate, 20 mL of water and 14 mg (0.06 mmol; 0.004 eq.) of ruthenium chloride. The reaction was exothermic, and the formation of the sodium iodate (NaIO₃) precipitate was observed very rapidly. After reaction for 1 hour, no further sulfite remained and only the sulfate (3) was observed on TLC. The reaction mixture was then filtered and diluted with 100 mL of CH₂Cl₂. The residual water from the reaction was removed and the organic phase was washed twice with 5% sodium bicarbonate (NaHCO₃) solution and then with distilled water. It was then dried over Na₂SO₄, filtered and concentrated to give a slightly brown solid.

This solid was dissolved in a minimum amount of CH₂Cl₂ in the presence of active charcoal, and filtered off on silica. The silica was rinsed with 300 mL of CH₂Cl₂. The brown impurities, containing the ruthenium salts, remained at the surface. The white solid obtained was then used in step 2) without further purification.

Yield: 84% over two steps.

Rf: 0.48 (PE/EtOAc 7/3 v/v).

MS: (ESI⁺/MeOH) m/z: 297 [M+H]⁺, 319 [M+Na]⁺.

¹H NMR (400.13 MHz, acetone-d₆) δ ppm: 1.38 and 1.53 (2s, 6H, H_{2′}); 3.46 (s, 3H, OCH₃); 4.17 (td, 1H, J₅₋₄=J_{5-6b}=10.6 Hz, J₅₋₆ₐ=5.5 Hz, H₅); 4.32 (dd, 1H, J₂₋₃=5.6 Hz, J₂₋₁=0.4 Hz, H₂); 4.42 (dd, 1H, J₃₋₂=5.6 Hz, J₃₋₄=7.7 Hz, H₃); 4.59 (dd, 1H, J₄₋₃=7.8 Hz, J₄₋₅=10.4 Hz, H₄); 4.64 (t, 1H, J_{6b-5}=10.7 Hz, J_{6b-6a}=−10.7 Hz, H_{6b}); 4.87 (dd, 1H, J₆ₐ₋₅=5.5 Hz, J_{6a-6b}=−10.5 Hz, H₆ₐ); 5.01 (d, 1H, J₁₋₂=0.5 Hz, H₁).

¹³C NMR (100.62 MHz, CDCl₃) δ ppm: 26.4 and 28.3 (2C, C_{2′}); 56.1 (1C, OCH₃); 58.9 (1C, C₅); 72.3 (1C, C₆); 73.6 (1C, C₃); 76.3 (1C, C₂); 84.6 (1C, C₄); 99.4 (1C, C₁); 111.0 (1C, C_{1′}).

2) Second step: Preparation of methyl 6,7-dideoxy-7-sulfonato-α-D-mannoheptopyranoside 2 (Compound I-1)

### 2-a) Nucleophilic Attack

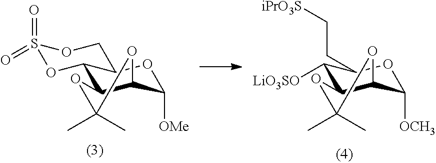

303 mg (2.19 mmol; 1.3 eq.) of isopropyl methyl sulfonate and 3 drops of 1,1-diphenylethylene (colored indicator) were dissolved in 2 mL of anhydrous tetrahydrofuran (THF) under argon. The mixture was cooled to a temperature of −70° C. and 2.19 mmol (1.3 eq.) of butyllithium were then added dropwise. A red color (due to 1,1-diphenylhexyllithium) gradually appeared. The addition of the butyllithium was stopped, and the dark red color persisted. After stirring for 5 minutes at the same temperature, 500 mg (1.69 mmol; 1 eq.) of compound (3) obtained above in step 1), predissolved in 3 mL of anhydrous THF, were added slowly to the mixture. The red color disappeared quickly. 580 μL (3.37 mmol; 2 eq.) of hexamethylphosphotriamide (HMPT) were then added. The mixture was then allowed to warm to room temperature. After 15 minutes, all the starting material was consumed. The reaction medium was then diluted with 20 mL of CH₂Cl₂. The product was extracted with 2×10 mL of distilled water. This aqueous phase was then washed with CH₂Cl₂ until the organic impurities, such as the diphenylethylene and the HMPT, were removed. After freeze-drying, the solid obtained was reused in reaction immediately, without further purification.

Yield: Quantitative.

Rf: 0.35 (CH₂Cl₂/MeOH 85/15 v/v).

The product revealed with anisaldehyde was khaki colored.

### 2-b) Deprotection

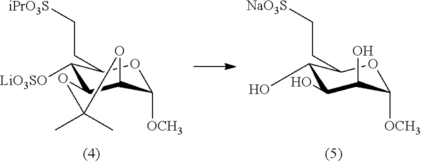

744 mg (1.69 mmol; 1 eq.) of methyl 6,7-dideoxy-7-sulfonate-4-lithiumsulfate-2,3-O-isopropylidene-α-D-mannoheptopyranoside (4) were dissolved in 10 ml of distilled water, followed by addition of 500 mg of a cation-exchange resin sold under the reference Amberlyst-15 H⁺ by the company Aldrich. After reaction for 3 hours, the resins were filtered off and the aqueous phase was freeze-dried. The solid obtained was purified by chromatography on silica gel, using as mobile phase an isopropanol (iPrOH)/aqueous ammonia (NH₄OH) mixture in an 8/2 (v/v) ratio to give a transparent foam. The exchange of the proton of the sulfonic acid with a sodium counterion was then performed, in water, using Dowex® Na⁺ resins sold by the company Dow Corning.

Yield: 95%.

Rf: 0.40 (iPrOH/NH₄OH 6/4 v/v).

MS (FAB⁺/NBA) m/z: 273 [M+H]⁺, 242 [M—OMe]⁺.

MS (FAB⁻/NBA) m/z: 271 [M−H]⁻.

¹H NMR (400.13 MHz, D₂O) δ ppm: 1.97 (m, 1H, H₇ₐ); 2.36 (m, 1H, H_{7b}); 2.99 (m, 1H, H₆ₐ); 3.13 (m, 1H, H_{6b}); 3.37 (s, 3H, OCH₃); 3.78 (m, 1H, H₅); 3.89-3.96 (m, 2H, H₃ and H₂); 4.45 (t, 1H, J₄₋₅=J₄₋₃=9.4 Hz, H₄); 4.70 (s, 1H, H₁).

¹³C NMR (100.62 MHz, D₂O) δ ppm: 26.8 (1C, C₇); 47.6 (1C, C₆); 55.4 (1C, OCH₃); 69.1 (1C, C₅); 69.9 (1C, C₃); 70.4 (1C, C₂), 79.0 (1C, C₄); 100.9 (1C, C₁).

### EXAMPLE 2

### Preparation of (Methyl 6,7-Dideoxy-α-D-Mannoheptopyranosine)uronic acid (compound of formula I-2)

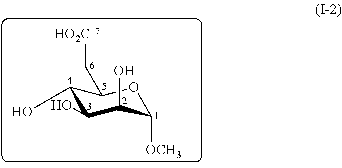

1) First step: Preparation of methyl 6-cyano-6-deoxy-4-O-sodiumsulfate-2,3-O-isopropylidene-α-D-mannopyranoside (compound 6).

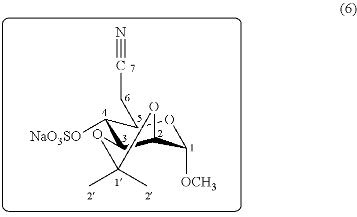

1 g (3.38 mmol; 1 eq.) of methyl 2,3-O-iso-propylidene-4,6-O-(cyclic sulfate)-α-D-mannopyranoside (compound 3) as obtained above after step 1) of example 1 was dissolved in 3 ml of dimethylformamide (DMF), followed by addition of 331 mg (6.75 mmol; 2 eq.) of sodium cyanide. The mixture was stirred magnetically at room temperature for 20 hours. The reaction medium was then diluted with 20 mL of 1% NaHCO₃ (to avoid any release of hydrogen cyanide (HCN)), and washed with 10 mL of CH₂Cl₂. The product was again extracted from the organic phase with 2×10 mL of distilled water. The combined aqueous phases were freeze-dried to give a slightly yellow solid, which was pure enough to be reused immediately in reaction. However, this product may also be purified by chromatography on silica gel with an elution gradient (CH₂Cl₂ to CH₂Cl₂/MeOH 91/9 v/v) to give a very slightly yellow foam.

Yield: Quantitative.

Rf: 0.49 (CH₂Cl₂/MeOH 85/15 v/v).

The product was revealed as burgundy with anisaldehyde.

MS (ESI⁺/MeOH) m/z: 384 [M+Na]⁺.

MS (ESI⁺/MeOH) m/z: 322 [M−Na]⁻.

¹H NMR (400.13 MHz, acetone-d₆) δ ppm: 1.24 and 1.41 (2s, 6H, H_{2′}); 2.76 (dd, 1H, J₆ₐ₋₅=9.3 Hz, J_{6a-6b}=−17.3 Hz, H₆ₐ); 3.18 (dd, 1H, J_{6b-5}=2.8 Hz, J_{6b-6a}=−17.3 Hz, H_{6b}); 3.46 (s, 3H, OCH₃); 3.86 (td, 1H, J₅₋₆ₐ=J₅₋₄=9.6 Hz, J_{5-6b}=2.8 Hz, H₅); 4.15 (d, 1H, J₂₋₃=7.4 Hz, H₂); 4.21 (dd, 1H, J₄₋₅=9.9 Hz, J₄₋₃=7.0 Hz, H₄); 4.44 (dd_{poorly resolved}, 1H, H₄); 4.93 (s, 1H, H₁).

¹³C NMR (100.62 MHz, acetone-d₆) δ ppm: 20.6 (1C, C₆); 25.5 and 27.1 (2C, C_{2′}); 54.5 (1C, OCH₃); 64.9 (1C, C₅); 75.6 (1C, C₂); 76.3 (1C, C₄); 76.9 (1C, C₃); 98.1 (1C, C₁); 109.8 (1C, C_{1′}); 118.1 (1C, C₇).

2) Second Step: Preparation of methyl 6-cyano-6-deoxy-α-D-mannopyranoside (compound 7)

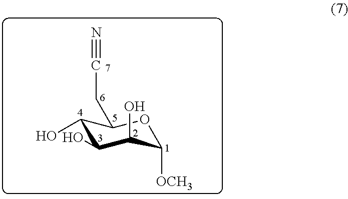

873 mg (2.53 mmol; 1 eq.) of methyl 6-deoxy-6-cyano-4-sodiumsulfate-2,3-O-isopropylidene-α-D-manno-heptopyranoside (6) obtained above in the preceding step were dissolved in 20 mL of a solution formed from a mixture of methanol (MeOH) and THF (1/1; v/v), and 1 g of Amberlyst-15 H⁺ was then added. After reaction for 1 hour 15 minutes, the resins were filtered off and the reaction medium was neutralized with 5% NaHCO₃ solution to pH 8. The organic solvents were removed on a rotary evaporator and the remaining water was freeze-dried. The mixture was taken up in MeOH, and the insoluble NaHCO₃ was filtered off. The product was then purified by chromatography on silica gel with an elution gradient (CH₂Cl₂ to CH₂Cl₂/MeOH 92/8 v/v) to give a white foam.

Yield: 72%.

Rf: 0.56 (CH₂Cl₂/MeOH 85/15 v/v).

MS: (ESI⁺/MeOH) m/z: 226 [M+Na]⁺, 242 [M+K]⁺, 429 [2M+Na]⁺.

¹H NMR (400.13 MHz, D₂O) δ ppm: 2.86 (dd, 1H, J₆ₐ₋₅=7.4 Hz, J_{6a-6b}=−17.3 Hz, H₆ₐ); 3.04 (dd, 1H, J_{6b-5}=3.6 Hz, J_{6b-6a}=−17.3 Hz, H_{6b}); 3.44 (s, 3H, OCH₃); 3.60 (t, 1H, J₄₋₅=J₄₋₃=9.7 Hz, H₄); 3.76 (dd, 1H, J₃₋₄=9.6 Hz, J₃₋₂=3.4 Hz, H₃); 3.84 (ddd, 1H, J₅₋₆ₐ=7.1 Hz, J_{5-6b}=3.2 Hz, J₅₋₄=10.1 Hz, H₅); 3.96 (dd, 1H, J₂₋₃=3.4 Hz, J₂₋₁=1.7 Hz, H₂); 4.78 (d, 1H, J₁₋₂=1.5 Hz, H₁).

¹³C NMR (100.62 MHz, D₂O) δ ppm: 51.4 (1C, C₆); 55.2 (1C, OCH₃); 67.8 (1C, C₅); 70.2 (1C, C₂); 70.7 (1C, C₃); 71.6 (1C, C₄); 101.4 (1C, C₁).

3) Third Step: Preparation of (Methyl 6,7-Dideoxy-α-D-mannoheptopyranoside)uronic acid (I-2)

200 mg (0.98 mmol; 1 eq.) of methyl 6-deoxy-6-cyano-α-D-mannoheptopyranoside (7) obtained above in the preceding step were dissolved in 2 mL of aqueous 30% hydrogen peroxide (H₂O₂) solution, followed by addition of 60 mg (1.46 mmol, 1.5 eq.) of sodium hydroxide (NaOH). The solution was left at room temperature. After 12 hours and then 24 hours of reaction, a further 1 mL of hydrogen peroxide solution and 30 mg of sodium hydroxide were added to the reaction medium.

After 48 hours, the reaction medium was neutralized with Amberlyst H⁺ resins and then filtered and freeze-dried. The product obtained was then purified by chromatography on silica gel with an elution gradient (CH₂Cl₂ to CH₂Cl₂/MeOH 85/15 v/v).

Rf: 0.25 (isopropanol/NH₄OH 85/15 v/v).

Yield: 80%.

MS: (ESI⁺/MeOH) m/z: 245 [M+Na]⁺.

MS: (ESI⁻/MeOH) m/z: 221 [M−H]⁻.

### EXAMPLE 3

### Preparation of methyl 6-deoxy-6-malonate-α-D-Mannopyranoside (Compound I-3)

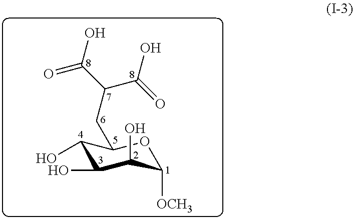

1) First Step: Preparation of methyl 2,3,4-tri-O-benzyl-6-deoxy-6-[bis(2,2,2-trifluoroethyl)malonate]-α-D-mannopyranoside (Compound 8)

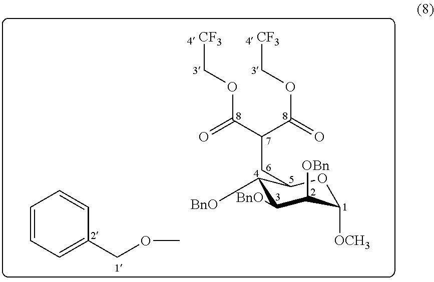

765 mg (1.65 mmol; 1 eq.) of methyl 2,3,4-tri-O-benzyl-α-D-mannopyranoside, 530 mg (1.98 mmol; 1.2 eq.) of bis(2,2,2-trifluoroethyl)malonate and 866 mg (3.3 mmol; 2 eq.) of triphenylphosphine were dissolved in 10 mL of toluene. 833 mg (3.3 mmol; 2 eq.) of 1,1′-(azodicarbonyl)dipiperidine (ADDP) were then added portionwise (over 30 minutes). The mixture was stirred magnetically at room temperature and the reaction was monitored by TLC (Et₂O/PE 4/6 v/v). After 48 hours, the reaction medium was filtered on silica, concentrated and deposited directly on a column. The product was purified by chromatography on silica gel with an elution gradient (PE to PE/Et₂O 85/15 v/v) to give a colorless oil.

Yield: 55%.

Rf: 0.79 (Et₂O/PE 6/4 v/v).

MS: (ESI⁺/MeOH) m/z: 713 [M+Na]⁺.

MS: (ESI⁻/MeOH) m/z: 737 [M−H]⁻.

¹H NMR (400.13 MHz, CDCl₃) δ ppm: 2.45 (ddd, 1H, J₆ₐ₋₅=10.0 Hz, J_{6a-6b}=−14.4 Hz, J₆ₐ₋₇=4.9 Hz, H₆ₐ); 2.87 (ddd, 1H, J_{6b-5}=2.6 Hz, J_{6b-6a}=−14.1 Hz, J_{6b-7}=9.0 Hz, H_{6b}); 3.51 (s, 3H, OCH₃); 3.84 (td, 1H, J₅₋₄=J₅₋₆ₐ=9.7 Hz, J_{5-6b}=2.6 Hz, H₅); 3.96 (t, 1H, J₄₋₃=J₄₋₃=9.3 Hz, H₄); 4.02 (dd, 1H, J₂₋₁=1.9 Hz, J₂₋₃=2.9 Hz, H₂); 4.11 (dd, 1H, J₃₋₂=3.1 Hz, J₃₋₄=9.2 Hz, H₃); 4.11 (dd, 1H, J₇₋₆ₐ=5.0 Hz, J_{7-6b}=9.2 Hz, H₇); 4.72 (m, 4H, H_{3′}); 4.84 (s, 2H, H_{1′}); 4.87 (d, 1H, J₁₋₂=1.7 Hz, H₁); ν₀=4.96 (ABq, 2H, ν_{A}=4.93, ν_{B}=4.99, Δν=24.8 Hz, J_{AB}=12.2 Hz, H_{1′}); ν₀=5.06 (ABq, 2H, ν_{A}=4.90, ν_{B}=5.21, Δν=121.8 Hz, J_{AB}=11.0 Hz, H_{1′}); 7.50-7.62 (m, 15H, H_{Ph}).

¹³C NMR (100.62 MHz, CDCl₃) δ ppm: 31.5 (1C, C₆); 48.4 (1C, C₇); 55.3 (1C, OCH₃); 61.5 (q, 1C, J_{C—F}=37.2 Hz, O_{3′}) 69.5 (1C, C₅); 72.6, 73.4 and 75.7 (3C, C_{1′}); 75.1 (1C, C₂); 78.7 (1C, C₄); 80.5 (1C, C₃); 99.7 (1C, C₁); 123.1 (q, 1C, J_{C—F}=276.9 Hz, C_{4′}); 127.3-128.9 (15C, CH_{Ph}); 138.7, 138.8 and 138.9 (3C, CIV_{Ph}); 167.4 and 167.7 (2C, C₈).

¹⁹F NMR (188.31 MHz, CDCl₃) δ ppm: 74.14 (dd, J_{F—H}=8.5 Hz).

2) Second Step: Preparation of methyl 6-deoxy-6-malonate-α-D-mannopyranoside (Compound I-3).
2-a) Hydrogenolysis of the benzyl groups

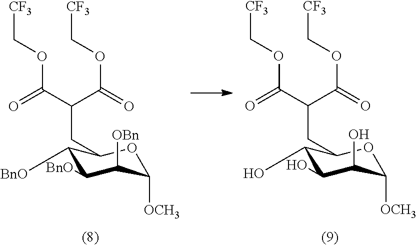

380 mg (0.53 mmol; 1 eq.) of methyl 6-deoxy-6-[bis(2,2,2-trifluoroethyl)malonate]-2,3,4-tri-O-benzyl-α-D-mannopyranoside (8) as obtained above in the preceding step were dissolved in 20 mL of MeOH, followed by addition of 130 mg of palladium-on-charcoal (Pd/C). The reaction medium was placed under a hydrogen atmosphere for 12 hours and then filtered through silica and concentrated to give a white foam, which was reused directly in reaction.

Yield: 90%.

Rf: 0.34 (Et₂O).

### 2-b) Hydrolysis of the Malonate Unit

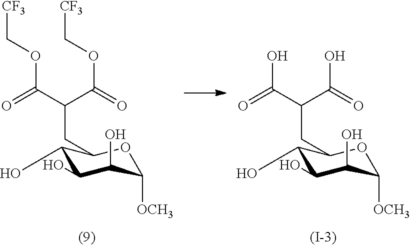

211 mg of methyl 6-deoxy-6-[bis(2,2,2-trifluoroethyl)malonate]-α-D-mannopyranoside (9) were dissolved in 5 mL of saturated ammoniacal methanol solution and left for 5 hours at 5° C. The reaction medium was then concentrated, and the product was then purified by chromatography on silica gel with an elution gradient (CH₂Cl₂/MeOH 9/1 v/v to CH₂Cl₂/MeOH 65/45 v/v) to give a white solid.

Yield: 90%.

Rf: 0.28 (CH₂Cl₂/MeOH 75/25 v/v).

MS: (ESI⁻/MeOH) m/z: 279 (M−H]⁻.

¹H NMR (400.13 MHz, D₂O) δ ppm: 1.96 (m, 1H, H₆ₐ); 2.49 (m, 1H, H_{6b}); 3.41 (s, 3H, OCH₃); 3.49 (m, 2H, H₃ and H₄); 3.61 (dd, 1H, J₇₋₆ₐ=5.75 Hz, J_{7-6b}=9.68 Hz, H₇); 3.71 (m, 1H, H₅); 3.92 (dd, 1H, J₂₋₁=1.68 Hz, J₂₋₃=3.26 Hz, H₂); 4.72 (s, 1H, H₁).

¹³C NMR (100.62 MHz, D₂O) δ ppm: 31.8 (1C, C₆); 49.9 (1C, C₇); 55.5 (1C, OCH₃); 70.2, 70.6 and 71.0 (4C, C₂, C₃, C₄ and C₅); 101.3 (1C, C₁); 174.1 and 174.9 (2C, C₈).

### EXAMPLE 4

### Demonstration of the Inhibitory Activity of M6P and of Three Derivatives Thereof on Angiogenesis—Comparative with Three D-Mannopyranoside Derivatives not Forming Part of the Invention

In this example, the activity of mannose-6-phosphate (M6P) and of the compounds of formulae (I-1), (I-2) and (I-3) as prepared in examples 1 to 3 above, respectively, on the inhibition of angiogenesis was studied, in comparison with three D-mannopyranoside (DM) derivatives having pro-angiogenic activity and thus not forming part of the invention (DM1: methyl 7-amino-6,7-dideoxy-α-D-mannopyranoside; DM2: methyl 6-azido-6-deoxy-α-D-mannopyranoside and DM3: methyl 7-disodiumphosphonato-6,7-dideoxy-α-D-mannohepto-pyranoside). This study was performed on chick embryos according to the method described by Ribatti D. et al., Nat. Protoc., 2006, 1(1), 85-91 with a few minor modifications.

### 1) Materials and Methods

This study was performed on the chorioallantoic membrane (CAM) of chick embryo. The CAM is an extra-embryonic membrane formed on the fourth day of incubation by fusion of the chorion and of the allantois. It allows gas exchange between the chick embryo and the extra-embryonic environment up to the time of birth. This CAM is composed of a very thick capillary network that forms a continuous surface in direct contact with the shell. Rapid capillary proliferation of this membrane continues up to the 11^{th} day; the mitotic index then decreases rapidly and the vascular system reaches its final organization on the 18^{th} day, just before birth (hatching on the 21^{st} day).

Fertilized eggs of a hen of the white Leghorn race were placed in an incubator from the start of embryogenesis, where they were kept under constant humidity at a temperature of 38° C. On the second day of incubation, a window was opened in the shell after removal of 2 to 3 mL of albumin in order to detach the CAM from the shell. The window was then sealed with adhesive tape and the egg was returned to the incubator to continue its development up to the date of the experiment. On the 7^{th} day, pieces of inert synthetic polymers (nitrocellulose filter disks 0.4 cm in diameter) were soaked with 20 μL of each of the solutions of the test compounds (6 mg/mL in PBS) and then positioned on the CAM. The impact of the test substances on the angiogenesis was then observed on the 12^{th} day and the quantitative evaluation of the pro- or anti-angiogenic response was estimated visually.

### 2) Results

The results obtained were photographed and are given in the attached FIG. 1, in which it may be observed that M6P and compounds (I-1), (I-2) and (I-3) have an inhibitory effect on the vascularization of chick embryos. Conversely, the derivatives DM1, DM2 and DM3 not forming part of the invention have an activating effect on the vascularization of the chick embryos. These results demonstrate that despite a very similar chemical structure, D-mannopyranose derivatives may have entirely opposite behavior on modulating angiogenesis.

These results as a whole clearly demonstrate that the compounds of formula (I) in accordance with the invention have an inhibitory action on angiogenesis.

1. A method of preparing a pharmaceutical composition, the method comprising:
combining, as an active principle, at least one compound of formula (I): 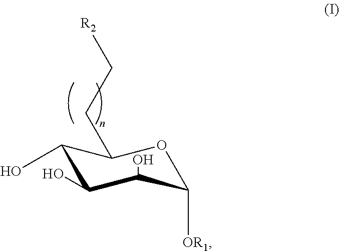
wherein:
R₁ represents a linear or branched C₁-C₄ alkyl radical; a second alkyl radical comprising at least one functional group selected from the group consisting of hydroxyl, amine, thiol, carboxyl, azide, and nitrile; a saturated or unsaturated C₃-C₆ hydrocarbon comprising ring; a second saturated or unsaturated C₃-C₆ hydrocarbon comprising ring comprising at least one functional group selected from the group consisting of hydroxyl, amine, C₁-C₄ alkyl, thiol, carboxyl, azide, and nitrile; or a saturated or unsaturated heterocycle comprising at least one heteroatoms selected from the group consisting of oxygen, nitrogen, and sulfur atoms;
n is 0 or 1,
R₂ is selected from the group consisting of (G₁), (G₂), (G₃) and (G₄): 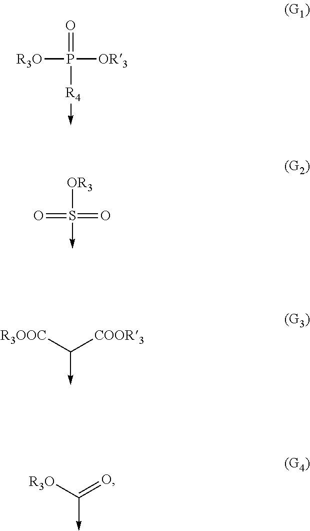
wherein:
R₃ and R′₃, which are optionally identical or different, represent a hydrogen or sodium atom;
R₄ represents an oxygen or sulfur atom, and
the arrow represents a point of attachment of the group to the carbon atom bearing R₂,
with a pharmaceutically acceptable excipient,
wherein the pharmaceutical composition is suitable for ligament regeneration and/or cartilage reconstruction.

2. The method of claim 1, wherein R₁ represents a methyl radical.

3. The method of in claim 1, wherein R₁ represents the C₁-C₄ alkyl radical or the second alkyl radical and the C₁-C₄ alkyl radical or the second alkyl radical is selected from the group consisting of a C₁-C₄ monohydroxyalkyl radical, a C₁-C₄-dihydroxyalkyl, a C₁-C₄ monoaminoalkyl radical, a C₁-C₄-diaminoalkyl radical, a C₁-C₄ monothialkyl radical, a C₁-C₄-dithioalkyl radical a C₁-C₄ monocarboxyalkyl radical, and a C₁-C₄-dicarboxyalkyl radical.

4. The method of claim 1, wherein R₁ represents the hydrocarbon comprising ring or the second hydrocarbon-comprising ring, and the hydrocarbon comprising ring or the second hydrocarbon-comprising ring is selected from the group consisting of cyclopropane, cyclobutane, cyclopentane, cyclohexane, phenyl, and benzyl.

5. The method of claim 1, wherein R₁ represents the heterocycle and the heterocycle is selected from the group consisting of oxadiazole, triazole, oxazole, isoxazole, imidazole, thiadiazole, pyrrole, tetrazole, furan, thiophene, pyrazole, pyrazoline, pyrazolidirie, thiazole, isothiazole, pyridine, pyrimidirie, piperidine, pyran, pyrazine, and pyridazine.

6. The method of claim 1, wherein, in the at least one compound of formula (I), when n=0, R₂ is G₃ or G₄, and when n=1, R₂ is G₁ or G₂.

7. The method of claim 1, wherein the at least one compound of formula (I) is selected from the group consisting of:
a compound in which R₂ represents a group G₁ or G₃, wherein R₃ and R′₃ are identical and represent a sodium atom, and
a compound in which R₂ represents a group G₂ or G₄, wherein R₃ represents a sodium atom.

8. The method of claim 1, wherein the at least one compound of formula (I) is selected from the group consisting of:
methyl D-mannopyranoside 6-phosphate;
methyl(disodium) D-mannopyranoside 6-phosphate;
methyl 6,7-dideoxy-7-sodiumsulfonato-D-manno-heptopyranoside;
(methyl 6,7-dideoxy-D-mannoheptopyranoside)-uronic acid; and
methyl 6-deoxy-6-malonate-D-mannopyranoside.

9. The method of claim 8, wherein the at least one compound of formula (I) is selected from the group consisting of
methyl 6,7-dideoxy-7-sodiumsulfonato-α-D-manno heptopyranoside,
methyl 6-deoxy-6-malonate-α-D-mannopyranoside, and
(methyl 6,7-dideoxy-α-D-mannoheptopyranosine)uronic acid.

10. The method of claim 1, wherein the pharmaceutical composition is in the form of a polymeric biomaterial comprising the at least one compound of formula (I).

11. The method of claim 2, wherein, in the at least one compound of formula (I), when n=0, R₂ is G₃ or O₄, and when n=1, R₂ is G₁ or G₂.

12. The method of claim 3, wherein, in the at least one compound of formula (I), when n=0, R₂ is G₃ or G₄, and when n=1, R₂ is G₁ or G₂.

13. The method of claim 4, wherein, in the at least one compound of formula (I), when n=0, R₂ is G₃ or G₄, and when n=1, R₂ is G₁ or G₂.

14. The method of claim 5, wherein, in the at least one compound of formula (I), when n=0, R₂ is O₃ or G₄, and when n=1, R₂ is G₁ or G₂.

15. The method of claim 2, wherein the at least one compound of formula (I) is selected from the group consisting of:
a compound in which R₂ represents a group G₁ or O₃, wherein R₃ and R′₃ are identical and represent a sodium atom, and
a compound in which R₂ represents a group G₂ or O₄, wherein R₃ represents a sodium atom.

16. The method of claim 3, wherein the at least one compound of formula (I) is selected from the group consisting of:
a compound in which R₂ represents a group G₁ or G₃, wherein R₃ and R′₃ are identical and represent a sodium atom, and
a compound in which R₂ represents a group G₂ or G₄, wherein R₃ represents a sodium atom.

17. The method of claim 4, wherein the at least one compound of formula (I) is selected from the group consisting of:
a compound in which R₂ represents a group G₁ or G₃, wherein R₃ and R′₃ are identical and represent a sodium atom, and
a compound in which R₂ represents a group G₂ or G₄, wherein R₃ represents a sodium atom.

18. The method of claim 5, wherein the at least one compound of formula (I) is selected from the group consisting of:
a compound in which R₂ represents a group G₁ or G₃, wherein R₃ and R′₃ are identical and represent a sodium atom, and
a compound in which R₂ represents a group G₂ or G₄, wherein R₃ represents a sodium atom.

19. The method of claim 6, wherein the at least one compound of formula (I) is selected from the group consisting of:
a compound in which R₂ represents a group G₁ or G₃, wherein R₃ and R′₃ are identical and represent a sodium atom, and
a compound in which R₂ represents a group G₂ or G₄ wherein R₃ represents a sodium atom.

20. The method of claim 2, wherein the at least one compound of formula (I) is selected from the group consisting of:
methyl D-mannopyranoside 6-phosphate;
methyl(disodium) D-mannopyranoside 6-phosphate;
methyl 6,7-dideoxy-7-sodiumsulfonato-D-manno-heptopyranoside;
(methyl 6,7-dideoxy-D-mannoheptopyranoside)-uronic acid; and
methyl 6-deoxy-6-malonate-D-mannopyranoside.
